(19) United States
(12) Patent Application Publication
(10) Pub. No.: US 2026/0061158 A1
(43) Pub. Date: Mar. 5, 2026

(54) MEDICAL DEVICE AND METHOD FOR MANUFACTURING MEDICAL DEVICE

(71) Applicant: ASAHI INTECC CO., LTD., Seto-shi (JP)

(72) Inventors: Makoto NISHIGISHI, Seto-shi (JP); Yoshiki KATO, Seto-shi (JP); Itsuki SUGIWAKA, Seto-shi (JP); Hirotomo SHIMIZU, Seto-shi (JP); Yusuke KIDO, Seto-shi (JP)

(73) Assignee: ASAHI INTECC CO., LTD., Seto-shi (JP)

(21) Appl. No.: 19/382,333

(22) Filed: Nov. 7, 2025

Related U.S. Application Data
(63) Continuation of application No. PCT/JP2023/018777, filed on May 19, 2023.

(51) Int. Cl. A61M 25/00

(52) U.S. Cl. CPC A61M 25/0009; A61M 2025/0037

(57) ABSTRACT

A medical device includes a first proximal end side tube that has a first bending rigidity and forms a part of a first lumen, a first distal end side tube that has a second bending rigidity lower than the first bending rigidity and forms a part of the first lumen, a second proximal end side tube that has a third bending rigidity and forms a part of the second lumen, and a second distal end side tube that has a fourth bending rigidity lower than the third bending rigidity and forms a part of the second lumen, and a distal end position of the second proximal end side tube is located closer to the distal end side than a distal end position of the first proximal end side tube in a longitudinal direction of the medical device.

### CROSS-REFERENCE TO RELATED APPLICATION

The present application claims priority to PCT/JP2023/018777, filed on May 19, 2023, the entire contents of which being incorporated herein by reference.

### TECHNICAL FIELD

The disclosed embodiments relate to a medical device and a method for manufacturing the medical device.

### BACKGROUND ART

There are known catheters used in percutaneous procedures. Here, in order to safely and efficiently perform a complicated percutaneous procedure, the procedure may be performed under the guidance of a sensor that acquires image information of a biological tissue by an ultrasonic wave. In such a case, a catheter having two lumens is used, a sensor is inserted into one lumen, and a therapeutic device is inserted into the other lumen so that the procedure is performed while using the sensor and the therapeutic device in combination. In order to improve safety and operability, such a catheter typically has a structure in which the distal end side is thin and flexible and the proximal end side is thick and highly rigid.

For example, Patent Literature 1 discloses a device including a support tube having a distal end side portion made of only a flexible resin and a proximal end side portion in which an inner resin layer and an outer resin layer are integrally laminated. For example, Patent Literature 2 discloses a medical elongated member which includes a first member and a second member that has an inner diameter larger than that of the first member and is provided on the proximal end side of the first member and in which a proximal end portion of the first member and a distal end portion of the second member are connected to each other with a connection member.

### CITATION LIST

### Patent Literature

Patent Literature 1: JP 2007-082707 A

Patent Literature 2: JP 2015-062532 A

### SUMMARY

### Technical Problems

However, in the device described in Patent Literature 1, there is an issue in that no consideration is given to the rigidity gap at the boundary between the distal end side portion configured to be flexible and the proximal end side portion configured to have a high rigidity. In addition, the elongated member described in Patent Literature 2 has only a single lumen and is used only for performing a treatment in an airway of the lung, and a multi-lumen configuration for realizing a procedure under the guide of a sensor is not considered at all. Such an issue is not limited to the vascular system and is common to medical devices inserted into various organs in a human body, such as a lymphatic system, a biliary system, a urinary system, an airway system, a digestive system, a secretory gland, and a reproductive organ.

The disclosed embodiments have been made in order to solve at least a part of the above-described issue and is directed to realizing gradual changes in rigidity in a medical device in which at least two or more lumens are formed by different tubes provided on the distal end side and the proximal end side.

### Solutions to Problems

The disclosed embodiments have been made to solve at least a part of the above-described and other issues and can be realized as the following aspects.

According to an aspect of the disclosed embodiments, a medical device is provided. The medical device includes a first proximal end side tube that has a first bending rigidity and forms a part of a first lumen, a first distal end side tube that has a second bending rigidity lower than the first bending rigidity, is provided closer to a distal end side than the first proximal end side tube, has a proximal end portion thereof joined to a distal end portion of the first proximal end side tube, and forms a part of the first lumen, a second proximal end side tube that has a third bending rigidity and forms a part of the second lumen, and a second distal end side tube that has a fourth bending rigidity lower than the third bending rigidity, is provided closer to the distal end side than the second proximal end side tube, has a proximal end portion thereof joined to a distal end portion of the second proximal end side tube, and forms a part of the second lumen, and a distal end position of the second proximal end side tube is located closer to the distal end side than a distal end position of the first proximal end side tube in a longitudinal direction of the medical device.

It should be noted that the disclosed embodiments can be realized in various modes and, for example, can be realized in modes such as a medical device, a medical tube, a catheter, and manufacturing methods thereof.

### BRIEF DESCRIPTION OF DRAWINGS

FIG. 1 is an explanatory view illustrating a configuration of a medical device.

FIG. 2 is an explanatory view illustrating a configuration of the medical device.

FIGS. 3A to 3E are transverse sectional views of a catheter.

FIG. 4 is a diagram illustrating a method of using the catheter.

FIG. 5 is a diagram illustrating a method of using the catheter.

FIG. 6 is an enlarged view of the catheter near a tube joint part.

FIGS. 7A and 7B are transverse sectional views of the catheter.

FIGS. 8A to 8C are diagrams illustrating a method for manufacturing the catheter.

FIGS. 9A to 9C are diagrams illustrating a method for manufacturing the catheter.

FIGS. 10A to 10C are diagrams illustrating a method for manufacturing a catheter according to a second embodiment.

FIG. 11 is an enlarged view of the vicinity of a tube joint part in a catheter according to a third embodiment.

FIGS. 12A and 12B are diagrams illustrating a method for manufacturing a catheter according to a fourth embodiment.

FIG. 13 is an enlarged view of the vicinity of a tube joint part in a catheter according to a fifth embodiment.

### DETAILED DESCRIPTION

### First Embodiment

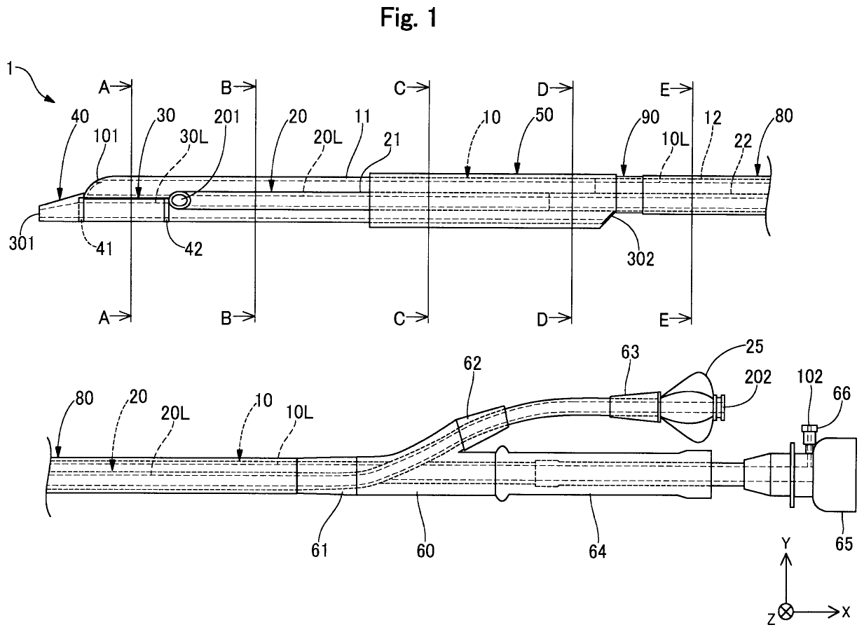

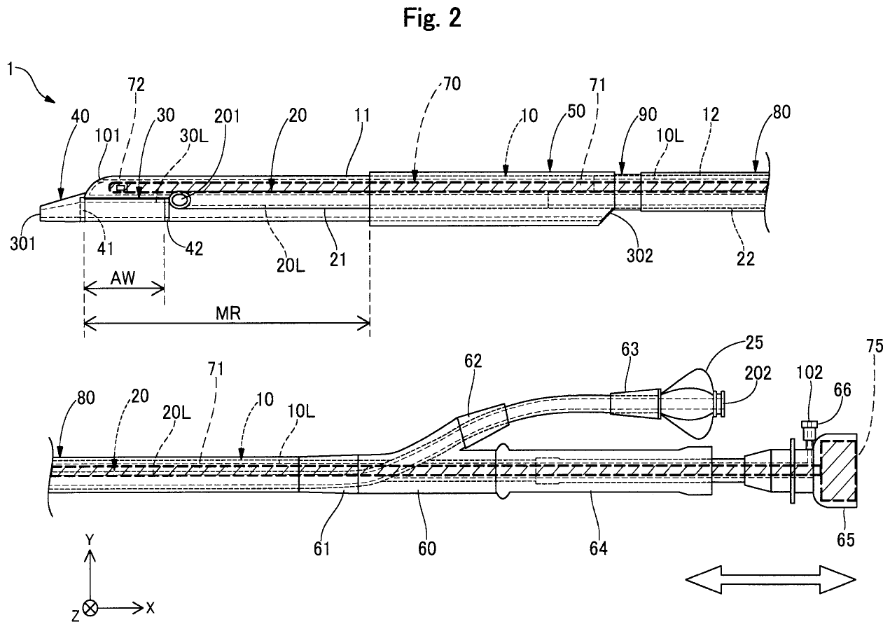

FIGS. 1 and 2 are explanatory views illustrating a configuration of a medical device 1. As will be detailed, the medical device is configured to provide a gradual transition in rigidity from a distal end to a proximal end. The medical device 1 according to the present embodiment is a catheter. Hereinafter, the medical device 1 is also referred to as the “catheter 1”. The catheter 1 is used to treat a lesion in a living body lumen such as a CTO occurring in a blood vessel. As illustrated in FIGS. 1 and 2, the catheter 1 includes a sensor tube 10, an OTW (Over The Wire) tube 20, an RX (Rapid Exchange) tube 30, a distal tip 40, a first marker 41, a second marker 42, a first outer tube 50, a branch connector 60, a first reinforcing member 61 to a third reinforcing member 63, a cylindrical member 64, a connector 65, a connector 25, a sensor 70, a second outer tube 80, and a heat-shrinkable tube 90. The sensor tube 10 is also referred to as the “medical device” or “medical tube”. The OTW tube 20 is also referred to as the “medical device” or “medical tube”.

In FIG. 1, the sensor 70 is not illustrated in order to explain the configurations of the tube and the lumen in the tube. In FIG. 2, the sensor 70 incorporated in a sensor lumen 10L in the sensor tube 10 is indicated by a broken line and hatched with oblique lines.

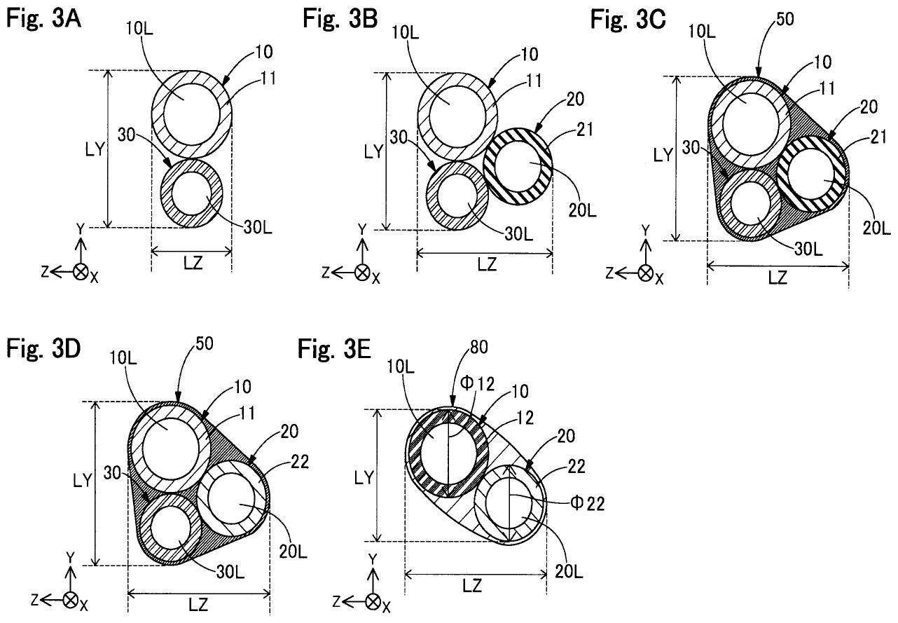

In FIGS. 1 and 2, for convenience of description, the relative ratio of the size of each constituent member includes a portion different from the actual portion. Further, a part of each constituent member includes an exaggerated portion. FIGS. 1 and 2 illustrate the XYZ axes orthogonal to each other. The X-axis corresponds to the longitudinal direction of the catheter 1, the Y-axis corresponds to the height direction of the catheter 1, and the Z-axis corresponds to the width direction of the catheter 1. The left side (−X-axis direction) of FIGS. 1 and 2 is referred to as the “distal end side” of the catheter 1 and each constituent member, and the right side (+X-axis direction) of FIGS. 1 and 2 is referred to as the “proximal end side” of the catheter 1 and each constituent member. In addition, among both ends of the catheter 1 and each constituent member in the longitudinal direction (X-axis direction), one end located on the distal end side is referred to as the “distal end”, and the other end located on the proximal end side is referred to as the “proximal end”. The distal end and the vicinity thereof are referred to as the “distal end portion”, and the proximal end and the vicinity thereof are referred to as the “proximal end portion”. The distal end side is inserted into a living body, and the proximal end side is operated by a professional such as a doctor. These points are common to FIGS. 3A to 3E and subsequent drawings.

FIGS. 3A to 3E are transverse sectional views of the catheter 1. FIG. 3A illustrates a transverse section of the catheter 1 taken along the line A-A in FIG. 1. FIG. 3B illustrates a transverse section of the catheter 1 taken along the line B-B in FIG. 1. FIG. 3C illustrates a transverse section of the catheter 1 taken along the line C-C in FIG. 1. FIG. 3D illustrates a transverse section of the catheter 1 taken along the line D-D in FIG. 1. FIG. 3E illustrates a transverse section of the catheter 1 taken along the line E-E in FIG. 1. Hereinafter, the configuration of the catheter 1 will be described with reference to FIGS. 1 to 3E.

The sensor tube 10 is a hollow cylindrical member (tubular body) having an elongated outer shape. The sensor tube 10 extends linearly along the longitudinal direction (X-axis direction) of the catheter 1 in parallel with the OTW tube 20 and the RX tube 30.

The sensor lumen 10L (broken line) for accommodating the sensor 70 is formed inside the sensor tube 10. The sensor lumen 10L is a lumen for the sensor 70.

In the longitudinal direction of the catheter 1, the distal end of the sensor tube 10 is located at the same position as the distal end of the RX tube 30 or slightly closer to the proximal end side than the distal end of the RX tube 30. A distal end opening 101 communicating between the distal end of the sensor lumen 10L and the outside is formed at the distal end of the sensor tube 10. The distal end opening 101 is a fluid discharge port for bringing the inside of the sensor lumen 10L into a wet state. The proximal end of the sensor tube 10 is located closer to the proximal end side than the proximal end of the OTW lumen 20L and the proximal end of the RX tube 30 in the longitudinal direction of the catheter 1. The first reinforcing member 61, the branch connector 60, the cylindrical member 64, and the connector 65 are attached on the proximal end side of the sensor tube 10 from the distal end side toward the proximal end side. Details will be described below. A fluid supply portion 66 is attached to the connector 65, and a proximal end opening 102 communicating between the proximal end of the sensor lumen 10L and the outside is formed in the fluid supply portion 66. The proximal end opening 102 is a fluid supply port to the sensor lumen 10L.

As illustrated in FIG. 1, the sensor tube 10 includes a first distal end side tube 11 provided on the distal end side and a first proximal end side tube 12 provided closer to the proximal end side than the first distal end side tube 11. Both the first distal end side tube 11 and the first proximal end side tube 12 are hollow cylindrical members (tubular bodies) having an elongated outer shape. The first distal end side tube 11 and the first proximal end side tube 12 are connected to each other inside the first outer tube 50 in the longitudinal direction. Specifically, both the first distal end side tube 11 and the first proximal end side tube 12 form a part of the sensor lumen 10L.

The OTW tube 20 is a hollow cylindrical member (tubular body) having an elongated outer shape. On the distal end side of the branch connector 60, the OTW tube 20 extends linearly along the longitudinal direction of the catheter 1 in parallel with the sensor tube 10 and the RX tube 30. An OTW lumen 20L (broken line) for accommodating a therapeutic device (for example, a plasma guide wire or a penetration guide wire) is formed inside the OTW tube 20. The OTW lumen 20L has no proximal end opening in a portion that is located in the living body lumen when the catheter 1 is used. The OTW lumen 20L is an over-the-wire (OTW) type lumen.

The distal end of the OTW tube 20 is located closer to the proximal end side than the distal end of the sensor tube 10 and the distal end of the RX tube 30 in the longitudinal direction of the catheter 1. A distal end opening 201 communicating between the distal end of the OTW lumen 20L and the outside is formed at the distal end of the OTW tube 20. The distal end opening 201 is a device projection port for projecting a therapeutic device toward a living tissue. Since the distal end portion of the OTW tube 20 is obliquely cut, the distal end opening 201 is oriented in a direction intersecting with the longitudinal direction of the catheter 1. Thus, when the catheter 1 is used, the therapeutic device can easily reach the living tissue present around the catheter 1. The proximal end of the OTW tube 20 is located closer to the distal end side than the proximal end of the sensor tube 10 and closer to the proximal end side than the proximal end of the RX tube 30 in the longitudinal direction of the catheter 1. The first reinforcing member 61, the branch connector 60, the second reinforcing member 62, the third reinforcing member 63, and the connector 25 are attached on the proximal end side of the OTW tube 20 from the distal end side toward the proximal end side. Details will be described below. A proximal end opening 202 communicating between the proximal end of the OTW lumen 20L and the outside is formed in the connector 25. The proximal end opening 202 is a device insertion port for inserting a therapeutic device into the OTW lumen 20L.

As illustrated in FIG. 1, the OTW tube 20 includes a second distal end side tube 21 provided on the distal end side and a second proximal end side tube 22 provided closer to the proximal end side than the second distal end side tube 21. Both the second distal end side tube 21 and the second proximal end side tube 22 are hollow cylindrical members (tubular bodies) having an elongated outer shape. The second distal end side tube 21 and the second proximal end side tube 22 are connected to each other inside the first outer tube 50 in the longitudinal direction. Specifically, both the second distal end side tube 21 and the second proximal end side tube 22 form a part of the OTW lumen 20L.

The RX tube 30 is a hollow cylindrical member (tubular body) having an elongated outer shape. The RX tube 30 extends linearly along the longitudinal direction of the catheter 1 in parallel with the sensor tube 10 and the OTW tube 20. An RX lumen 30L (broken line) for accommodating a work hose wire is formed inside the RX tube 30.

The distal end of the RX tube 30 is located at the same position as the distal end of the sensor tube 10 or slightly closer to the distal end side than the distal end of the sensor tube 10 in the longitudinal direction of the catheter 1. A hollow distal tip 40 is joined to the distal end portion of the RX tube 30. A distal end opening 301 communicating between the distal end of the RX lumen 30L and the outside is formed at the distal end of the distal tip 40. The distal end opening 301 is a wire insertion port for inserting a work hose wire into the RX lumen 30L. The proximal end of the RX tube 30 is located closer to the distal end side than the proximal end of the sensor tube 10 and the proximal end of the OTW tube 20 in the longitudinal direction of the catheter 1. A proximal end opening 302 communicating between the proximal end of the RX lumen 30L and the outside is formed at the proximal end of the RX tube 30. The proximal end opening 302 is a wire drawing port for drawing out the work hose wire to the outside. Since the proximal end of the RX tube 30 is obliquely cut, the proximal end opening 302 is oriented in a direction intersecting with the longitudinal direction of the catheter 1. Thus, when the catheter 1 is used, the work hose wire can be easily drawn out from the proximal end opening 302.

The distal tip 40 has radiopacity and is a cylindrical member in which the outer diameter expands from the distal end side toward the proximal end side. The distal tip 40 is joined to the distal end portion of the RX tube 30 and is thus located at the distal end of the catheter 1 and advances in the living body lumen prior to the other members. The inner cavity of the distal tip 40 communicates with the RX lumen 30L of the RX tube 30 and, as described above, the distal end opening 301 communicating between the distal end of the RX lumen 30L and the outside is formed at the distal end of the distal tip 40.

The first marker 41 and the second marker 42 are annular members having radiopacity. The first marker 41 is provided such that the proximal end of the first marker 41 and the proximal end of the distal tip 40 are at the same position in the longitudinal direction of the catheter 1. The first marker 41 is embedded between the outer peripheral surface of the RX tube 30 and the inner peripheral surface of the distal tip 40. The second marker 42 is provided such that the proximal end of the second marker 42 and the distal end of the distal end opening 201 are at the same position in the longitudinal direction of the catheter 1. The second marker 42 is joined to the outer peripheral surface of the RX tube 30. For joining the first marker 41 and the second marker 42, for example, joining between resins by thermal melting or joining with an adhesive such as an epoxy-based adhesive can be employed. Further, the second marker 42 may be colored to be directly visible by the professional. As described above, by arranging the first marker 41 and the second marker 42 on the RX tube 30, it is possible to prevent the first marker 41 and the second marker 42 from interfering with sensing (acquisition of image information) by the sensor 70.

As illustrated in FIG. 3A, in the transverse section taken along the line A-A, the sensor tube 10 (specifically, the first distal end side tube 11) and the RX tube 30 are provided, and the outer peripheral surfaces thereof are joined to each other. As illustrated in FIG. 3B, in the transverse section taken along the line B-B, the sensor tube 10 (specifically, the first distal end side tube 11), the OTW tube 20 (specifically, the second distal end side tube 21), and the RX tube 30 are provided, and the outer peripheral surfaces thereof are joined to each other. As illustrated in FIG. 3C, in the transverse section taken along the line C-C, the sensor tube 10 (specifically, the first distal end side tube 11), the OTW tube 20 (specifically, the second distal end side tube 21), and the RX tube 30 are covered with the first outer tube 50. Specifically, the outer peripheral surfaces of the three tubes 10, 20, and 30 are covered with the first outer tube 50 formed by melting, and thus the three tubes 10, 20, and 30 are integrally fixed. As illustrated in FIG. 3D, in the transverse section taken along the line D-D, the sensor tube 10 (specifically, the first distal end side tube 11), the OTW tube 20 (specifically, the second proximal end side tube 22), and the RX tube 30 are covered with the first outer tube 50, as in FIG. 3C. As illustrated in FIG. 3E, in the transverse section taken along the line E-E, the sensor tube 10 (specifically, the first proximal end side tube 12) and the OTW tube 20 (specifically, the second proximal end side tube 22) are covered with the second outer tube 80. Specifically, the outer peripheral surfaces of the two tubes 10 and 20 are covered with the second outer tube 80 formed by melting, and thus the two tubes 10 and 20 are integrally fixed.

In the A-A transverse section and the B-B transverse section, the sensor tube 10, the OTW tube 20, and the RX tube 30 may be joined together by using any joining material such as an epoxy-based adhesive, or may be welded by heat. In the A-A transverse section, the B-B transverse section, the C-C transverse section, and the D-D transverse section, a height LY of the catheter 1 is greater than a width LZ of the catheter 1. On the other hand, in the E-E transverse section, the height LY of the catheter 1 is smaller than the width LZ of the catheter 1. As illustrated in FIGS. 3A to 3E, the size relationship among the outer diameters of the three tubes 10, 20, and 30 is the outer diameter of the sensor tube 10>the outer diameter of the OTW tube 20>the outer diameter of the RX tube 30. In addition, the size relationship among the inner diameters (lumens) of the three tubes 10, 20, and 30 is the inner diameter of the sensor lumen 10L>the inner diameter of the OTW lumen 20L>the inner diameter of the RX lumen 30L. However, the size relationship among the outer diameters and the inner diameters is merely an example, and may be arbitrarily changed.

The A-A transverse section and the B-B transverse section, in other words, the outer shape of the catheter 1 closer to the distal end side than the first outer tube 50 is the shape along the contour of the two tubes 10 and 30 (or the three tubes 10, 20, and 30) arranged adjacent to each other, and a constricted portion (recess portion) is formed in the adjacent portion of each tube. The C-C transverse section and the D-D transverse section, in other words, the outer shape of the catheter 1 in the portion covered with the first outer tube 50 is a triangular shape with round corners (a round-corner triangular shape). The E-E transverse section, in other words, the outer shape of the catheter 1 in the portion covered with the second outer tube 80 is elliptical.

With reference back to FIG. 1, the description will be continued. The three tubes 10, 20, and 30 (specifically, the sensor tube 10, the OTW tube 20, and the RX tube 30) are fixed by the three tubes 90, 50, and 80.

The heat-shrinkable tube 90 is provided between the first outer tube 50 and the second outer tube 80 in the longitudinal direction of the catheter 1. The heat-shrinkable tube 90 covers the sensor tube 10 (specifically, a part of the first proximal end side tube 12 on the distal end side) and the OTW tube 20 (specifically, a part of the second proximal end side tube 22 on the distal end side) to bundle the two tubes 10 and 20. The heat-shrinkable tube 90 does not cover the RX tube 30, and the RX tube 30 is provided along the outer peripheral surface of the heat-shrinkable tube 90 in a state where the outer peripheral surface of the heat-shrinkable tube 90 and the outer peripheral surface of the RX tube 30 are in contact with each other. The distal end of the heat-shrinkable tube 90 is located closer to the proximal end side than the distal end of the first outer tube 50 and closer to the distal end side than the proximal end opening 302. That is, the distal end portion of the heat-shrinkable tube 90 is covered with the first outer tube 50. The proximal end of the heat-shrinkable tube 90 is located closer to the proximal end side than the distal end of the second outer tube 80 and closer to the distal end side than the first reinforcing member 61. That is, the proximal end portion of the heat-shrinkable tube 90 is covered with the second outer tube 80. In other words, an intermediate portion of the heat-shrinkable tube 90 in the longitudinal direction of the catheter 1 is not covered with the first outer tube 50 or the second outer tube 80.

The first outer tube 50 is located closer to the distal end side than the heat-shrinkable tube 90 in the longitudinal direction of the catheter 1. The first outer tube 50 is provided in a section which is closer to the proximal end side than the distal end opening 201 and in which the three tubes 10, 20, and 30 extend side by side. In the example of FIG. 1, the distal end of the first outer tube 50 is located near the center between the distal end opening 201 and the proximal end opening 302. The proximal end of the first outer tube 50 is located near the proximal end of the proximal end opening 302. As described above, the first outer tube 50 may be provided at a position away from the distal end opening 201 toward the proximal end side. This can prevent the first outer tube 50 from interfering with sensing (acquisition of image information) by the sensor 70 inserted into the sensor lumen 10L. The first outer tube 50 covers and fixes the distal end portion of the heat-shrinkable tube 90, the sensor tube 10 (specifically, a part of the first distal end side tube 11 on the proximal end side) exposed from the distal end of the heat-shrinkable tube 90, the OTW tube 20 (specifically, a part of the second distal end side tube 21 on the proximal end side and a part of the second proximal end side tube 22 on the distal end side) exposed from the distal end of the heat-shrinkable tube 90, and the RX tube 30. As illustrated in FIGS. 3C and 3D, the first outer tube 50 has a triangular outer shape with round corners and has a thick portion formed by melting along the outer peripheral surfaces of the three tubes 10, 20, and 30.

The second outer tube 80 is located closer to the proximal end side than the heat-shrinkable tube 90 in the longitudinal direction of the catheter 1. The second outer tube 80 is provided in a section which is closer to the proximal end side than the proximal end opening 302 and in which the two tubes 10 and 20 extend side by side. In the example of FIG. 1, the distal end of the second outer tube 80 is located at a portion slightly away from the proximal end opening 302 toward the proximal end side. The proximal end of the second outer tube 80 is located inside the first reinforcing member 61. The second outer tube 80 covers and fixes the proximal end portion of the heat-shrinkable tube 90, the sensor tube 10 (specifically, a part of the first proximal end side tube 12 on the proximal end side) exposed from the proximal end of the heat-shrinkable tube 90, and the OTW tube 20 (specifically, a part of the second proximal end side tube 22) exposed from the proximal end of the heat-shrinkable tube 90. As illustrated in FIG. 3E, the second outer tube 80 has an elliptical outer shape and has a thick portion formed by melting along the outer peripheral surfaces of the two tubes 10 and 20.

The branch connector 60 is a member having a bifurcated inner cavity and is provided on the proximal end side of the catheter 1. The OTW tube 20 is inserted into one inner cavity of the branch connector 60. The sensor tube 10 is inserted into the other inner cavity of the branch connector 60. The first reinforcing member 61 is a hollow cylindrical member provided closer to the distal end side than the branch connector 60. The first reinforcing member 61 covers the outer periphery of the second outer tube 80 in which the sensor tube 10 and the OTW tube 20 are bundled, thereby reinforcing the distal end side of the branch connector 60.

The second reinforcing member 62 is a hollow cylindrical member provided closer to the proximal end side than one branch of the branch connector 60. The second reinforcing member 62 covers the outer periphery of the OTW tube 20 inserted into the branch connector 60, thereby reinforcing the proximal end side of the branch connector 60. The third reinforcing member 63 is a hollow cylindrical member provided closer to the distal end side than the connector 25. The third reinforcing member 63 covers the outer periphery of the OTW tube 20 inserted into the connector 25, thereby reinforcing the distal end side of the connector 25. The connector 25 is a member joined to the proximal end portion of the OTW tube 20. The connector 25 includes a pair of blade portions to be gripped by the professional. The proximal end opening 202 (device insertion port) communicating between the proximal end of the OTW lumen 20L and the outside is formed at the proximal end of the connector 25.

The cylindrical member 64 is a hollow cylindrical member provided closer to the proximal end side than the other branch of the branch connector 60. The cylindrical member 64 covers the outer periphery of the sensor tube 10 inserted into the branch connector 60, thereby reinforcing the proximal end side of the branch connector 60. The connector 65 is a member joined to the proximal end portion of the sensor tube 10. A housing for accommodating a connection terminal 75 of the sensor 70 is provided on the proximal end side of the connector 65. The fluid supply portion 66, in which the proximal end opening 102 communicating between the proximal end of the sensor lumen 10L and the outside is formed, is provided on the outer peripheral surface of the connector 65.

The sensor 70 (FIG. 2) is an imaging sensor that acquires image information. As illustrated in FIG. 2, the sensor 70 includes a main body part 71, a probe 72, and a connection terminal 75. The main body part 71 is an elongated member extending along the longitudinal direction of the catheter 1. A driving cable (coaxial line) that electrically connects the probe 72 and the connection terminal 75 is incorporated inside the main body part 71. The probe 72 includes an ultrasonic probe (also referred to as an ultrasonic vibrator, a piezoelectric body, an ultrasonic transmission/reception element, or an ultrasonic element) that transmits an ultrasonic wave toward a living tissue and receives the ultrasonic wave propagated through the living tissue and reflected. The probe 72 is also referred to as an imaging core or a transducer. The connection terminal 75 is a terminal that electrically connects the sensor 70 to a console terminal provided outside. The connection terminal 75 is provided at the proximal end of the main body part 71 and is accommodated in the housing of the connector 65.

The sensor 70 is electrically connected to an external console terminal via the connection terminal 75, receives the power supplied from the console terminal, and outputs a detection signal by the probe 72 to the console terminal. Thus, the console terminal can display the image information based on the detection signal of the probe 72. As illustrated in FIG. 2, the sensor 70 is fixed to the connector 65. Further, as indicated by the white arrow in FIG. 2, the professional grips the connector 65 and slides the connector 65 in the front-rear direction (the direction of the white arrow) and thus can move the position of the probe 72 of the sensor 70 within a range MR from the distal end of the sensor lumen 10L to the distal end of the first outer tube 50, in other words, within the predetermined range MR including the distal end opening 201. Hereinafter, the range MR is also referred to as the “movable range MR”. Further, a portion of the catheter 1 which is particularly suitable for sensing (acquisition of image information) by the sensor 70 is also referred to as an “acoustic window AW”. As illustrated in FIG. 2, the acoustic window AW is a section between the first marker 41 and the second marker 42 in the catheter 1.

The first distal end side tube 11 of the sensor tube 10, the second distal end side tube 21 of the OTW tube 20, and the RX tube 30 can be formed of a flexible material, for example, a thermoplastic resin such as a polyethylene resin, a polypropylene resin, or polyurethane, polyvinyl chloride, ethylene-vinyl acetate copolymers, cross-linked ethylene-vinyl acetate copolymers, polyamide elastomer, polyolefin elastomer, polyurethane elastomer, silicone rubber, or latex rubber. The first distal end side tube 11 of the sensor tube 10, the second distal end side tube 21 of the OTW tube 20, and the RX tube 30 may be formed of the same material or different materials.

The first proximal end side tube 12 of the sensor tube 10 and the second proximal end side tube 22 of the OTW tube 20 can be formed of, for example, a resin having a high rigidity such as a nylon resin, a polyester resin, or a PEEK resin. The melting points of the first proximal end side tube 12 of the sensor tube 10 and the second proximal end side tube 22 of the OTW tube 20 are higher than the melting points of the above-described tubes 11, 21, and 30. The first proximal end side tube 12 of the sensor tube 10 and the second proximal end side tube 22 of the OTW tube 20 may be formed of the same material or different materials.

In the catheter 1 according to the present embodiment, by providing the section in which a part of the RX tube 30 having flexibility on the proximal end side is overlapped with the first proximal end side tube 12 and the second proximal end side tube 22 having a high rigidity (FIG. 1), gradual changes in rigidity of the catheter 1 is achieved (a rigidity gap of the catheter 1 is reduced). As a result, kink of the catheter 1 can be suppressed. Further, any one or more of the first distal end side tube 11 and the first proximal end side tube 12 of the sensor tube 10, the second distal end side tube 21 and the second proximal end side tube 22 of the OTW tube 20, and the RX tube 30 may have a multilayer structure in which tubes made of different materials are stacked.

The distal tip 40, the first marker 41, and the second marker 42 can be formed of a resin material or a metal material having radiopacity. For example, when a radiopaque resin material is used, it can be formed by mixing a radiopaque material such as bismuth trioxide, tungsten, or barium sulfate with a polyamide resin, a polyolefin resin, a polyester resin, a polyurethane resin, a silicone resin, or a fluororesin. For example, when a radiopaque metal material is used, it can be formed of gold, platinum, tungsten, or an alloy containing these elements (for example, a platinum-nickel alloy). The distal tip 40, the first marker 41, and the second marker 42 may be formed of the same material or different materials.

The branch connector 60, the first reinforcing member 61 to the third reinforcing member 63, the cylindrical member 64, the connector 65, and the connector 25 can be formed of a known resin material. The branch connector 60, the first reinforcing member 61 to the third reinforcing member 63, the cylindrical member 64, the connector 65, and the connector 25 may be formed of the same material or different materials.

The heat-shrinkable tube 90 is formed of a nylon-based elastomer resin (for example, polyamide elastomer) having thermoplasticity. The heat-shrinkable tube 90 has a property of shrinking without melting when heated. In addition, the heat-shrinkable tube 90 has improved adhesiveness (property of easily sticking to another substance) at the time of heating compared to that at the time of non-heating. The heat-shrinkable tube 90 may be formed of polyolefin, FEP (Fluorinated Ethylene Propylene), or silicone.

The first outer tube 50 and the second outer tube 80 are formed of a nylon-based elastomer resin having thermoplasticity. Unlike the heat-shrinkable tube 90, the first outer tube 50 and the second outer tube 80 have a property of melting when heated. In the example according to the present embodiment, a resin having lower shore hardness than the second outer tube 80 is used for the first outer tube 50. However, the first outer tube 50 and the second outer tube 80 may be formed of the same material or may be formed of different materials.

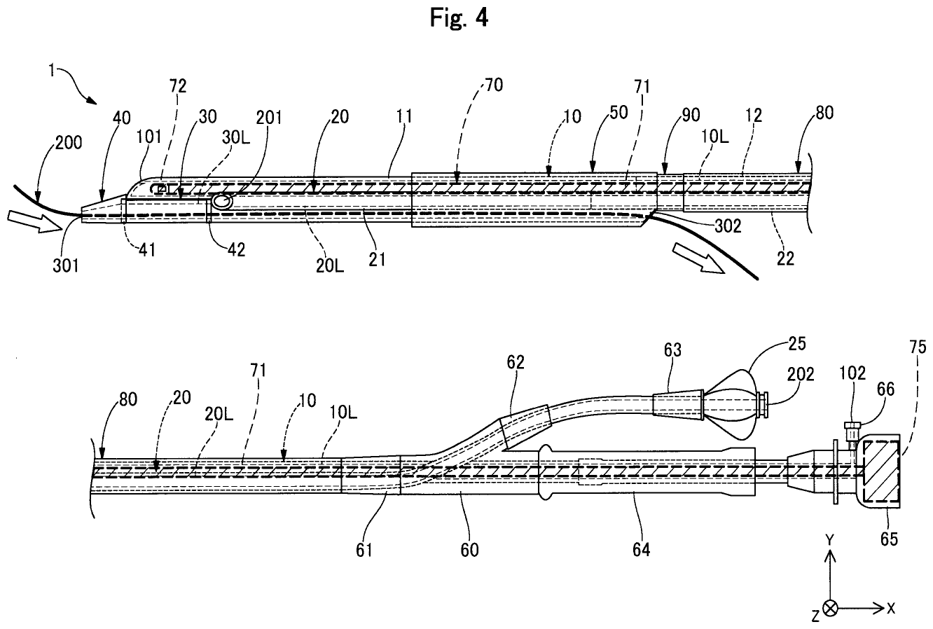

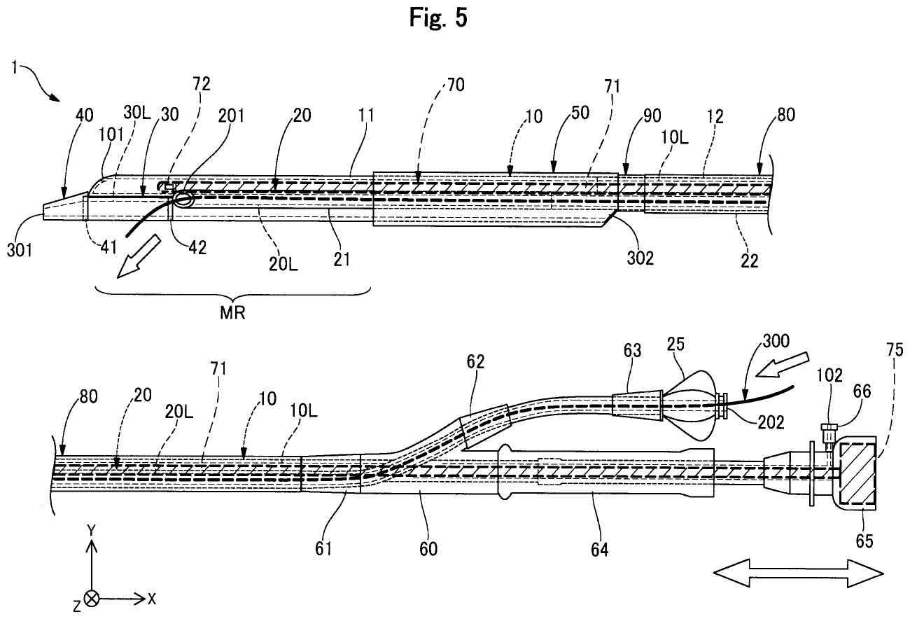

FIGS. 4 and 5 are diagrams illustrating a method of using the catheter 1. In the following steps a1 to a6, the case of recanalization of a CTO (lesion) generated in a blood vessel by the forward approach will be exemplified. However, the catheter 1 may be used in an inverse approach and may be used for operation other than recanalization of a CTO.
(a1) The professional inserts the work hose wire 200 into the blood vessel and delivers the distal end portion of the work hose wire 200 to the vicinity of the CTO. (a2) The professional inserts the proximal end portion of the work hose wire 200 from the distal end opening 301 of the catheter 1, passes it through the RX lumen 30L, and draws it out from the proximal end opening 302 of the catheter 1 (FIG. 4). (a3) The professional pushes the catheter 1 into the blood vessel along the work hose wire 200 and delivers the distal end portion of the catheter 1 to the vicinity of the CTO. In the step a3, the catheter 1 may be delivered to the vicinity of the CTO by passing the catheter 1 through a guiding catheter inserted into the blood vessel in advance along the work hose wire 200. (a4) While adjusting the position of the probe 72 of the sensor 70 within the movable range MR by gripping the connector 65 and sliding the connector 65 in the front-rear direction (FIG. 5: the direction of the white arrow), the professional checks the image displayed on the console terminal and thus matches the positions and orientations of the CTO and the distal end opening 201. The position means the position in the extending direction of the blood vessel, and the orientation means the orientation in the circumferential direction of the inner wall of the blood vessel. (a5) The professional inserts the distal end portion of a therapeutic device 300 from the proximal end opening 202 of the catheter 1, inserts the distal end portion into the OTW lumen 20L, and protrudes the distal end portion from the distal end opening 201 of the catheter 1 (FIG. 5). (a6) The professional treats the CTO using the therapeutic device 300 while adjusting the position of the probe 72 of the sensor 70 within the movable range MR as necessary and checking the image displayed on the console terminal. As described above, any device such as a plasma guide wire or a penetration guide wire can be used as the therapeutic device 300.

The sensor tube 10, the OTW tube 20, and the RX tube 30 are also collectively referred to as a “shaft”. The sensor tube 10 corresponds to a “first tube”, and the sensor lumen 10L corresponds to a “first lumen”. The OTW tube 20 corresponds to a “second tube”, and the OTW lumen 20L corresponds to a “second lumen”. The RX tube 30 corresponds to a “third tube”, and the RX lumen 30L corresponds to a “third lumen”. The heat-shrinkable tube 90 corresponds to a “heat-shrinkable tube”. According to the present embodiment, “the same” and “equal” are not limited to a case of an exact match, and have a meaning of allowing a difference due to a manufacturing error or the like. In addition, “constant” is synonymous with “substantially constant”, and means substantially constant while allowing a deviation due to a manufacturing error or the like.

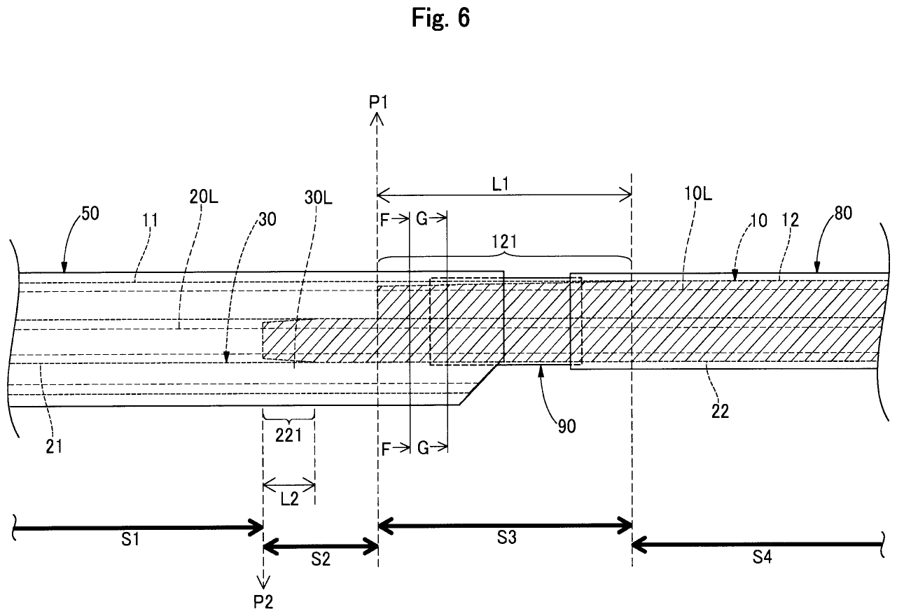

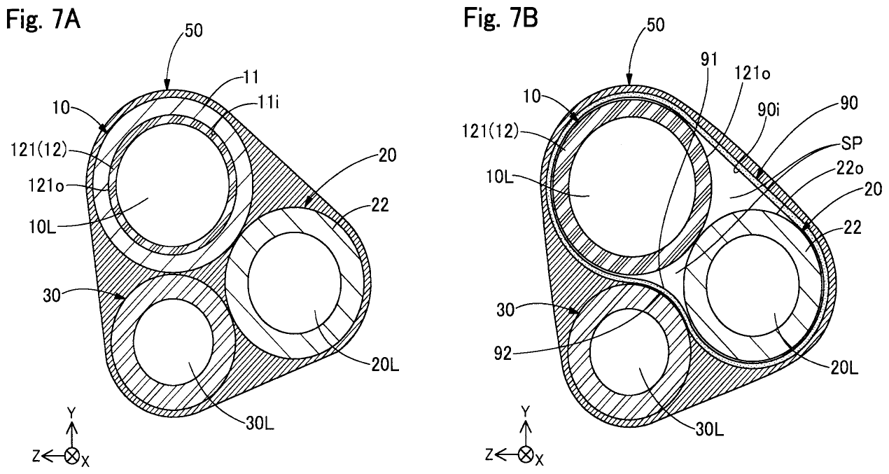

FIG. 6 is an enlarged view of the catheter 1 near a tube joint part. In FIG. 6, for convenience of description, the first proximal end side tube 12 and the second proximal end side tube 22 are hatched with oblique lines. FIGS. 7A and 7B are transverse sectional views of the catheter 1. FIG. 7A illustrates a transverse section taken along the line F-F in FIG. 6. FIG. 7B illustrates a transverse section taken along the line G-G in FIG. 6. The joining between the first and second distal end side tubes 11, 21 and the first and second proximal end side tubes 12, 22 will be further described with reference to FIGS. 6, 7A, and 7B.

In the sensor tube 10, the bending rigidity of the first proximal end side tube 12 is referred to as the “first bending rigidity”, and the bending rigidity of the first distal end side tube 11 is referred to as the “second bending rigidity”. In this case, the second bending rigidity is lower than the first bending rigidity. Therefore, the melting point of the first proximal end side tube 12 is higher than the melting point of the first distal end side tube 11. In addition, in the OTW tube 20, the bending rigidity of the second proximal end side tube 22 is referred to as the “third bending rigidity”, and the bending rigidity of the second distal end side tube 21 is referred to as the “fourth bending rigidity”. In this case, the fourth bending rigidity is lower than the third bending rigidity. Therefore, the melting point of the second proximal end side tube 22 is higher than the melting point of the second distal end side tube 21. The first to fourth bending rigidities can be arbitrarily determined as long as the above-described magnitude relationship is satisfied. In the example according to the present embodiment, the first bending rigidity and the third bending rigidity are the same, but they may be different. Further, the second bending rigidity and the fourth bending rigidity are the same, but may be different. This difference in bending rigidity may be achieved by forming the proximal end side tubes 12 and 22 from a high-rigidity material, e.g., PEEK resin, and forming the distal end side tubes 11 and 21 from a more flexible material, e.g., polyurethane, as described above.

In the sensor tube 10, the first distal end side tube 11 is provided closer to the distal end side than the first proximal end side tube 12, and the distal end portion of the first proximal end side tube 12 and the proximal end portion of the first distal end side tube 11 are joined to each other. Thus, the sensor tube 10 having the sensor lumen 10L is formed.

The distal end portion of the first proximal end side tube 12 is provided with a first tapered portion 121 in which the outer diameter of the first proximal end side tube 12 becomes smaller from the proximal end toward the distal end. In the first tapered portion 121, the wall thickness of the first proximal end side tube 12 gradually decreases toward the distal end side. The first tapered portion 121 is covered with the proximal end portion of the first distal end side tube 11. In other words, the first distal end side tube 11 and the first proximal end side tube 12 are provided to overlap with each other in the range where the first tapered portion 121 is provided. The first tapered portion 121 is also simply referred to as a “tapered portion”.

The first distal end side tube 11 and the first proximal end side tube 12 are joined to each other in an immiscible state at the portion where the first distal end side tube 11 and the first proximal end side tube 12 overlap with each other. Hereinafter, this portion is also referred to as the “first joint part”. As illustrated in FIG. 7A, at the first joint part, an outer peripheral surface 121o of the first tapered portion 121 is in contact with an inner peripheral surface 11i of the first distal end side tube 11. In the illustrated example, the outer peripheral surface 121o and the inner peripheral surface 11i are in contact with each other entirely in the circumferential direction; however, the outer peripheral surface 121o and the inner peripheral surface 11i may be in contact with each other only at a partial portion in the circumferential direction, and may have a portion where they are not in contact with each other.

In the OTW tube 20, the second distal end side tube 21 is provided closer to the distal end side than the second proximal end side tube 22, and the distal end portion of the second proximal end side tube 22 and the proximal end portion of the second distal end side tube 21 are joined to each other. Thus, the OTW tube 20 having the OTW lumen 20L is configured. The distal end portion of the second proximal end side tube 22 is provided with a second tapered portion 221 in which the outer diameter of the second proximal end side tube 22 becomes smaller from the proximal end toward the distal end. In the second tapered portion 221, the wall thickness of the second proximal end side tube 22 gradually decreases toward the distal end side. The second tapered portion 221 is covered with the proximal end portion of the second distal end side tube 21. In other words, the second distal end side tube 21 and the second proximal end side tube 22 are provided to overlap with each other in the range where the second tapered portion 221 is provided.

The second distal end side tube 21 and the second proximal end side tube 22 are joined to each other in an immiscible state at the portion where the second distal end side tube 21 and the second proximal end side tube 22 overlap with each other. Hereinafter, this portion is also referred to as a “second joint part”. Similarly to the first tapered portion 121 described in FIG. 7A, the outer peripheral surface of the second tapered portion 221 is in contact with the inner peripheral surface of the second distal end side tube 21 at the second joint part.

As illustrated in FIG. 6, in the longitudinal direction of the catheter 1, a distal end position P2 of the second proximal end side tube 22 is located closer to the distal end side than a distal end position P1 of the first proximal end side tube 12. Specifically, the first joint part and the second joint part are shifted in the longitudinal direction of the catheter 1. In other words, the first joint part and the second joint part are at different positions in the longitudinal direction of the catheter 1. This intentional misalignment or staggering the joints means the first joint part (where tube 11 joins tube 12) and the second joint part (where tube 21 joins tube 22) are shifted relative to one another along the catheter's longitudinal axis. This staggered configuration aids in creating a gradual transition in bending rigidity. In addition, in the longitudinal direction of the catheter 1, the proximal end position of the second tapered portion 221 is located closer to the distal end side than the distal end position P1 of the first proximal end side tube 12. Specifically, the first joint part and the second joint part are at different positions and are at positions that do not overlap with each other in the longitudinal direction of the catheter 1. Furthermore, in the longitudinal direction of the catheter 1, a length L1 of the first tapered portion 121 is longer than a length L2 of the second tapered portion 221. The lengths L1 and L2 can be arbitrarily determined as long as the above-described magnitude relationship is satisfied.

As illustrated in FIG. 3E, an outer diameter φ12 of the first proximal end side tube 12 is larger than an outer diameter φ22 of the second proximal end side tube 22. Here, the outer diameter φ12 means the outer diameter of the first proximal end side tube 12 closer to the proximal end side than the first tapered portion 121. Similarly, the outer diameter φ22 means the outer diameter of the second proximal end side tube 22 closer to the proximal end side than the second tapered portion 221.

As illustrated in FIG. 6, the proximal end of the RX tube 30 (third tube) is located between the distal end and the proximal end of the first tapered portion 121. In other words, the proximal end of the RX tube 30 is located within the range where the first tapered portion 121 is provided.

As illustrated in FIG. 6, the heat-shrinkable tube 90 bundles a part of the distal end side of the first proximal end side tube 12 of the sensor tube 10 and a part of the distal end side of the second proximal end side tube 22 of the OTW tube 20. The heat-shrinkable tube 90 is provided between the distal end and the proximal end of the first tapered portion 121. In other words, the heat-shrinkable tube 90 is provided within the range in which the first tapered portion 121 is provided.

As illustrated in FIG. 7B, the first tapered portion 121 of the first proximal end side tube 12 and the second proximal end side tube 22 are covered with the heat-shrinkable tube 90 in a state where parts of the outer peripheral surfaces 121o and 22o are in contact with each other. A space SP is formed in the region surrounded by the outer peripheral surfaces 121o and 22o and an inner peripheral surface 90i of the heat-shrinkable tube 90. The space SP exists on both sides of a contact portion between the tubes 12 and 22. The heat-shrinkable tube 90 includes a protruding portion 91 that protrudes toward one of the spaces SP (i.e., the space SP closer to the RX tube 30). On the opposite side (outer peripheral surface side) of the protruding portion 91 of the heat-shrinkable tube 90, a recess portion 92 is formed in which the heat-shrinkable tube 90 is recessed toward the contact portion side of the tubes 12 and 22. The RX tube 30 (third tube) is provided in contact with the recess portion 92 of the outer peripheral surface of the heat-shrinkable tube 90. This configuration can reduce the outer diameter of the catheter 1 in the G-G transverse section illustrated in FIG. 7B.

At the portion where the heat-shrinkable tube 90 and the first outer tube 50 overlap with each other, the first outer tube 50 covers the heat-shrinkable tube 90 and the RX tube 30 provided in contact with the outer peripheral surface of the heat-shrinkable tube 90 (FIG. 7B). The outer peripheral surface of the heat-shrinkable tube 90 and the outer peripheral surface of the RX tube 30 are integrally fixed to each other with the first outer tube 50 formed by melting.

Here, sections S1 to S4 along the longitudinal direction of the catheter 1 are defined. As illustrated in FIG. 6, the section S1 is a section from the distal end of the second distal end side tube 21 to the distal end P2 of the second proximal end side tube 22. The section S2 is a section from the distal end P2 of the second proximal end side tube 22 to the distal end P1 of the first proximal end side tube 12. The section S3 is a section from the distal end to the proximal end of the first tapered portion 121, in other words, the section in which the first tapered portion 121 is provided. The section S4 is a section from the proximal end of the first tapered portion 121 to the proximal end of the first proximal end side tube 12. In other words, the sections S2 and S3 serve as a transition region between the section S1 and the section S4.

Since the section S1 is a section in which the first distal end side tube 11 and the second distal end side tube 21 having the relatively low second and fourth bending rigidities are present, the rigidity of the catheter 1 is relatively low. The section S4 is a section in which the first proximal end side tube 12 and the second proximal end side tube 22 having the relatively high first and third bending rigidities are present, and is a section in which no tapered portion is present in any of the first proximal end side tube 12 and the second proximal end side tube 22; therefore, the rigidity of the catheter 1 is relatively high. Since the section S2 is a section in which the first distal end side tube 11 having the relatively low second bending rigidity and the second proximal end side tube 22 having the relatively high third bending rigidity are present, the rigidity of the catheter 1 is higher than that of the section S1 and lower than that of the section S4. The section S3 is a section in which the first proximal end side tube 12 and the second proximal end side tube 22 having the relatively high first and third bending rigidities are present, but the first tapered portion 121 is formed in the first proximal end side tube 12; therefore, the rigidity of the catheter 1 is higher than that of the section S1 and the section S2 and is lower than that of the section S4. That is, in the sections S1 to S4 along the longitudinal direction of the catheter 1, the rigidities of the catheter 1 are S1<S2<S3<S4, creating a stepped, gradual increase in stiffness from the distal end toward the proximal end. This configuration mitigates abrupt changes in rigidity that could otherwise lead to kinking. As described above, in the catheter 1 according to the present embodiment, it is possible to realize gradual changes in rigidity from the distal end side toward the proximal end side of the catheter 1.

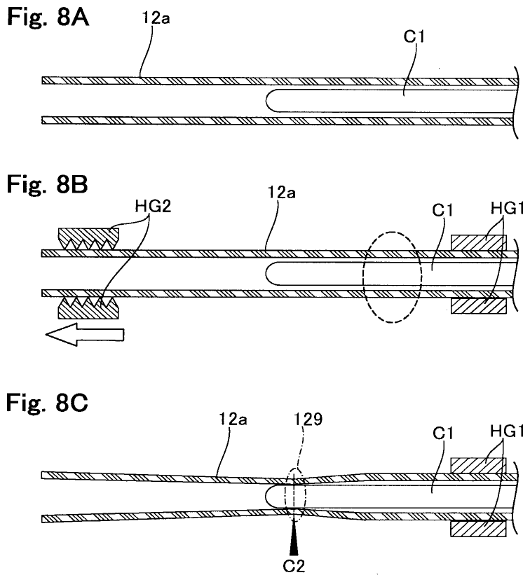

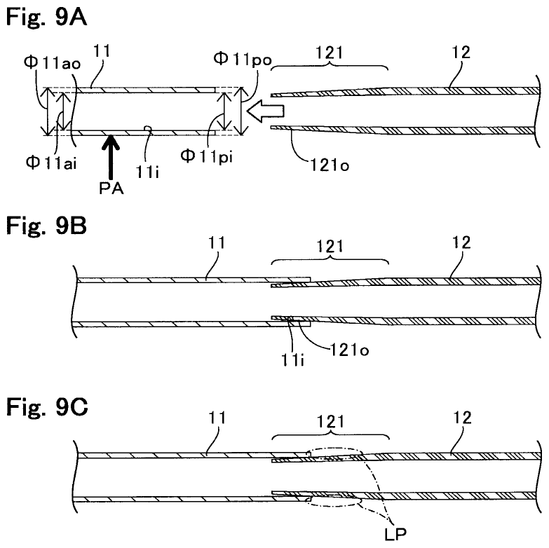

FIGS. 8A to 8C and 9A to 9C are diagrams illustrating a method for manufacturing the catheter 1. In the case described with reference to FIGS. 8A to 8C and 9A to 9C, the first distal end side tube 11 and the first proximal end side tube 12 are joined to form the sensor tube 10. However, the same method as in FIGS. 8A to 8C and 9A to 9C can be adopted when the second distal end side tube 21 and the second proximal end side tube 22 are joined to form the OTW tube 20. In addition, in FIGS. 8A to 8C, “a” is added to the end of the reference numeral of the tube which is the material of the first proximal end side tube 12.

FIG. 8A illustrates a step of placing a tube 12a in a constriction formation step.

As illustrated in FIG. 8A, the operator prepares the tube 12a, which is an elongated tubular body, and inserts a cored bar C1 into the tube 12a. At this time, the distal end position of the cored bar C1 is closer to the proximal end side than the distal end position of the tube 12a. According to the present embodiment, a PEEK tube is used as the tube 12a.

FIG. 8B illustrates a step of forming a constriction 129 in the constriction formation step. As illustrated in FIG. 8B, the operator holds a portion of the tube 12a away from the cored bar C1 to the proximal end side with a first chuck HG1 and holds the distal end portion of the tube 12a with a second chuck HG2. In this state, the operator stretches the tube 12a by pulling the second chuck HG2 in the direction of the white arrow while heating a part (broken line circle frame) of the tube 12a closer to the distal end side than the first chuck HG1 at a prescribed heating temperature. The heating temperature is a temperature at which the tube 12a is melted and deformed. In the example according to the present embodiment, the heating temperature is higher than the melting point of the first distal end side tube 11 by 100° C. or more. For example, a bench vise can be used as the first chuck HG1 and, for example, longnose pliers can be used as the second chuck HG2.

FIG. 8C illustrates a tapered portion formation step. As illustrated in FIG. 8C, by the above-described heating and stretching of the tube 12a, the constriction 129 surrounded by a dash-dotted line circle is formed at the distal end portion of the cored bar C1 in the tube 12a. The constriction 129 is a portion where the outer diameter of the tube 12a is the thinnest. Then, as illustrated in FIG. 8C, the operator cuts the tube 12a at the constriction 129 using a Feather cutter C2. Thus, the first proximal end side tube 12 having the first tapered portion 121 formed at the distal end portion can be formed (FIG. 9A).

FIG. 9A illustrates a state before insertion in an insertion step. As illustrated in FIG. 9A, the operator prepares the first distal end side tube 11. Regarding the first distal end side tube 11, the outer diameter at the proximal end is defined as an outer diameter φ11po, and the inner diameter at the proximal end is defined as an inner diameter φ11pi. With regard to the first distal end side tube 11, an outer diameter at a position PA (hereinafter, also referred to as the “prescribed position PA”) away from the proximal end of the first distal end side tube 11 by an arbitrary distance in the long axis direction (the longitudinal direction of the first distal end side tube 11) is defined as an outer diameter φ11ao, and the inner diameter at the prescribed position PA is defined as an inner diameter φ11ai. In this case, the outer diameter φ11po at the proximal end of the first distal end side tube 11 is substantially the same as the outer diameter φ11ao at the prescribed position PA. Further, the inner diameter φ11pi at the proximal end of the first distal end side tube 11 is substantially the same as the inner diameter φ11ai at the prescribed position PA. In other words, the outer diameter and the inner diameter of the first distal end side tube 11 are constant to the proximal end. As indicated by the white arrow in FIG. 9A, the operator inserts the first tapered portion 121 of the first proximal end side tube 12 into the first distal end side tube 11. In the illustrated example, a part of the first tapered portion 121 on the distal end side is inserted into the first distal end side tube 11.

FIG. 9B illustrates a state after insertion in the insertion step. As a result of FIG. 9A, as illustrated in FIG. 9B, the first distal end side tube 11 and the first proximal end side tube 12 are provided in a state where the outer peripheral surface 121o of the first tapered portion 121 of the first proximal end side tube 12 is in contact with the inner peripheral surface 11i of the first distal end side tube 11.

FIG. 9C illustrates a joining step. As illustrated in FIG. 9C, after inserting the first proximal end side tube 12 into the first distal end side tube 11, the operator applies any adhesive such as an epoxy-based adhesive to an outer peripheral surface of a step portion LP between the first distal end side tube 11 and the first proximal end side tube 12. The step portion LP is a portion which is closer to the proximal end side than the proximal end of the first distal end side tube 11 and in which the outer diameter of the first proximal end side tube 12 is reduced by the first tapered portion 121. The application of the adhesive to the step portion LP may be omitted. By the above-described procedure, the sensor tube 10 is prepared in which the distal end portion of the first proximal end side tube 12 and the proximal end portion of the first distal end side tube 11 are in contact with each other in an immiscible state and thus joined to each other.

Then, the operator prepares the OTW tube 20 by the same procedure as described above and then bundles the sensor tube 10 and the OTW tube 20 with the heat-shrinkable tube 90. Then, the operator arranges the RX tube 30 with respect to the tubes 10 and 20 bundled with the heat-shrinkable tube 90 and integrates these tubes by using the first outer tube 50. The sensor tube 10 (the first distal end side tube 11 and the first proximal end side tube 12) and the OTW tube 20 (the second distal end side tube 21 and the second proximal end side tube 22) are fixed by the first outer tube 50. As illustrated in FIG. 7A, the step portion LP in FIG. 9C is covered with the thick portion of the first outer tube 50, and therefore does not appear on the outer shape.

As described above, with the catheter 1 according to the first embodiment, as illustrated in FIG. 6, the distal end position P2 of the second proximal end side tube 22 is located closer to the distal end side than the distal end position P1 of the first proximal end side tube 12. Specifically, the first joint part where the first distal end side tube 11 and the first proximal end side tube 12 are joined to each other and the second joint part where the second distal end side tube 21 and the second proximal end side tube 22 are joined to each other are shifted in the longitudinal direction of the catheter 1 (medical device) (located at different positions). For this reason, as compared to a case where the first joint part and the second joint part are at the same position, it is possible to realize gradual changes in rigidity of the catheter 1 (FIG. 6: S1<S2<S3<S4), and it is possible to suppress the occurrence of kink in the catheter 1. As a result, when a pushing force is applied to the catheter 1 during use of the catheter 1, buckling of the catheter 1 can be suppressed.

In addition, with the catheter 1 according to the first embodiment, since the first tapered portion 121 is provided at the distal end portion of the first proximal end side tube 12, it is possible to easily connect the first proximal end side tube 12 to the first distal end side tube 11 with the first tapered portion 121. Similarly, since the second tapered portion 221 is provided at the distal end portion of the second proximal end side tube 22, the second proximal end side tube 22 can be easily connected to the second distal end side tube 21 with the second tapered portion 221. Further, since the length L1 of the first tapered portion 121 and the length L2 of the second tapered portion 221 are different from each other, gradual changes in rigidity can be further improved.

Further, with the catheter 1 according to the first embodiment, since the RX tube 30 is provided, the catheter 1 (medical device) can be configured to have the RX lumen 30L (third lumen).

Furthermore, with the catheter 1 according to the first embodiment, in the longitudinal direction of the catheter 1, the proximal end position of the second tapered portion 221 is located closer to the distal end side than the distal end position P1 of the first proximal end side tube 12. In other words, the first tapered portion 121 and the second tapered portion 221 do not overlap with each other in the longitudinal direction of the catheter 1 and are located at different positions. Therefore, as described in FIG. 6, it is possible to reduce the variation in the outer diameter between the first proximal end side tube 12 and the second proximal end side tube 22 in the section in the longitudinal direction of the catheter 1 in which the three tubes (specifically, the first proximal end side tube 12, the second proximal end side tube 22, and the RX tube 30 as the third tube) are present. As a result, as illustrated in FIG. 7B, it is possible to reduce deviation of the position of the RX tube 30 (the RX tube 30 is provided to be deviated with respect to any one of the first proximal end side tube 12 and the second proximal end side tube 22). In other words, for example, when the first proximal end side tube does not include the first tapered portion 121, the RX tube 30 may be provided to be deviated with respect to one of the first and second proximal end side tubes due to a difference in the outer diameter between the first proximal end side tube having a relatively large diameter and the second proximal end side tube having a relatively small diameter. In this regard, in the catheter 1 (medical device) according to the present embodiment, since the first proximal end side tube 12 includes the first tapered portion 121, as illustrated in FIG. 7B, such deviation can be suppressed.

Further, with the catheter 1 according to the first embodiment, as illustrated in FIG. 7B, by using the heat-shrinkable tube 90, the first proximal end side tube 12 and the second proximal end side tube 22 can be bundled without being bonded. In addition, as illustrated in FIG. 6, since the heat-shrinkable tube 90 is provided between the distal end and the proximal end of the first tapered portion 121, it is possible to suppress an increase in the outer diameter of the catheter 1 (medical device) due to the presence of the heat-shrinkable tube 90.

Furthermore, with the catheter 1 according to the first embodiment, since the melting point of the first proximal end side tube 12 is higher than the melting point of the first distal end side tube 11, it is possible to join tubes having different melting points to each other. Furthermore, with the catheter 1 according to the first embodiment, since the first proximal end side tube 12 and the first distal end side tube 11 are in contact with each other in an immiscible state, the tubes can be joined to each other without being compatibilized with each other.

With the method for manufacturing the catheter 1 according to the first embodiment, as illustrated in FIGS. 8A to 8C and 9A, the first proximal end side tube 12 and the first distal end side tube 11 can be joined to each other by using the first tapered portion 121 (tapered portion) formed at the distal end portion of the first proximal end side tube 12. Further, with the method for manufacturing the catheter 1 according to the first embodiment, as described in FIG. 8B, even when the melting points of the first proximal end side tube 12 and the first distal end side tube 11 are different from each other by 100° C. or more, the first proximal end side tube 12 and the first distal end side tube 11 can be joined to each other. Furthermore, with the method for manufacturing the catheter 1 according to the first embodiment, as described in FIGS. 9A and 9B, by using the first tapered portion 121, the first proximal end side tube 12 and the first distal end side tube 11 can be joined to each other without performing processing (for example, flaring processing) on the first proximal end side tube 12.

### Second Embodiment

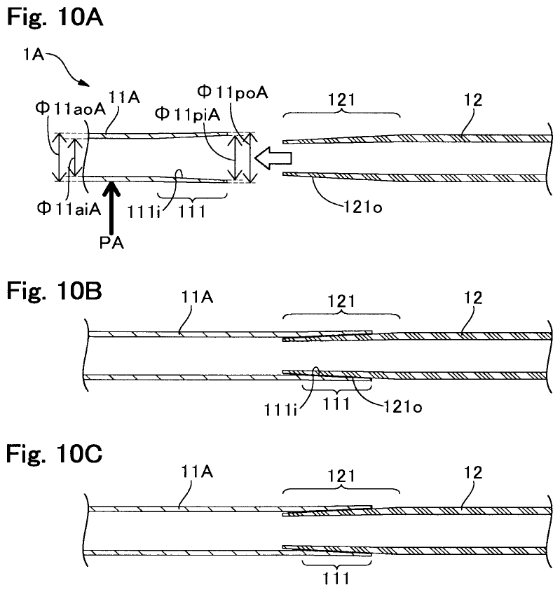

FIGS. 10A to 10C are diagrams illustrating a method for manufacturing a catheter 1A according to a second embodiment. The catheter 1A according to the second embodiment is different from that according to the first embodiment described in FIGS. 9A to 9C in the contents of the insertion step and the joining step. According to the second embodiment, the constriction formation step and the tapered portion formation step are the same as in the first embodiment.

FIG. 10A illustrates a state before insertion in the insertion step. As illustrated in FIG. 10A, the operator prepares a first distal end side tube 11A. Unlike the first distal end side tube 11 according to the first embodiment, a proximal end portion of the first distal end side tube 11A has undergone flaring processing. Specifically, an outer diameter φ11poA at the proximal end of the first distal end side tube 11A is larger than an outer diameter φ11aoA at the prescribed position PA. Further, an inner diameter φ11piA at the proximal end of the first distal end side tube 11A is larger than an inner diameter φ11aiA at the prescribed position PA. In other words, the outer diameter and the inner diameter of a part of the first distal end side tube 11A on the proximal end side gradually increase. The portion of the first distal end side tube 11A where the outer diameter φ11poA and the inner diameter φ11piA are enlarged is also referred to as a “flare portion 111”. As indicated by the white arrow in FIG. 10A, the operator inserts the first tapered portion 121 of the first proximal end side tube 12 into the flare portion 111 of the first distal end side tube 11A.

FIG. 10B illustrates a state after insertion in the insertion step. As a result of FIG. 10A, as illustrated in FIG. 10B, the first distal end side tube 11A and the first proximal end side tube 12 are provided in a state where the outer peripheral surface 121o of the first tapered portion 121 of the first proximal end side tube 12 is in contact with an inner peripheral surface 111i of the flare portion 111 of the first distal end side tube 11A. As illustrated, according to the present embodiment, since the proximal end portion of the first distal end side tube 11A is the flare portion 111, when the first proximal end side tube 12 is inserted into the first distal end side tube 11A, the step LP described in the first embodiment is not formed (or the step LP is reduced).

FIG. 10C illustrates a joining step. As illustrated in FIG. 10C, after inserting the first proximal end side tube 12 into the first distal end side tube 11A, the operator applies any adhesive such as an epoxy-based adhesive to the outer peripheral surface of the first proximal end side tube 12 located closer to the proximal end side than the first distal end side tube 11A. The subsequent procedure is the same as that described in the first embodiment.

As described above, the method for manufacturing the catheter 1A can be variously changed, and the occurrence of the step LP may be suppressed by forming the flare portion 111 at the proximal end portion of the first distal end side tube 11A. The above-described change is merely an example, and the method for manufacturing the catheter 1A can be changed in various ways. For example, the application of the adhesive in the joining step may be performed on the outer peripheral surface of the first tapered portion 121 of the first proximal end side tube 12. The catheter 1A and the method for manufacturing the catheter 1A according to the second embodiment described above can also achieve the same effects as those of the first embodiment described above.

### Third Embodiment

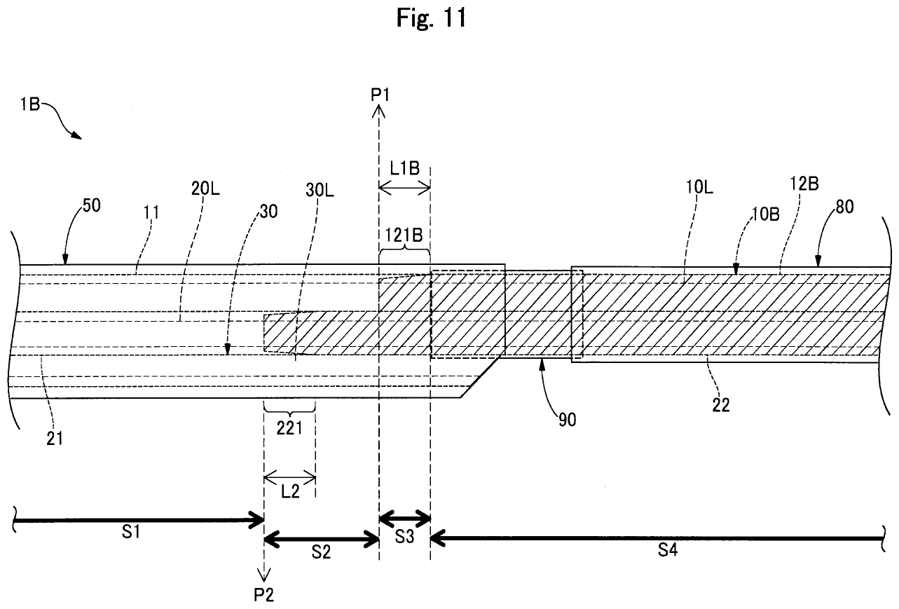

FIG. 11 is an enlarged view of the vicinity of a tube joint part in a catheter 1B according to a third embodiment. The catheter 1B according to the third embodiment includes a sensor tube 10B instead of the sensor tube 10 in the configuration described in the first embodiment. The sensor tube 10B includes a first proximal end side tube 12B including a first tapered portion 121B instead of the first proximal end side tube 12 described in the first embodiment.

A length L1B of the first tapered portion 121B in the longitudinal direction of the catheter 1B is shorter than the length L1 described in the first embodiment. In the illustrated example, the length L1B of the first tapered portion 121B is the same as the length L2 of the second tapered portion 221. As a result, in the catheter 1B, the proximal end of the RX tube 30 is located closer to the proximal end side than the first tapered portion 121B.

As described above, the configuration of the catheter 1B can be variously changed, and the length L1B of the first tapered portion 121B and the length L2 of the second tapered portion 221 can be arbitrarily changed. For example, the length L1B of the first tapered portion 121B may be shorter than the length L2 of the second tapered portion 221. One of the lengths L1B and L2 may be set to zero. In this case, one of the first proximal end side tube 12B and the second proximal end side tube 22 does not have a tapered portion. The catheter 1B according to the third embodiment described above can also achieve the same effects as those of the first embodiment described above.

### Fourth Embodiment

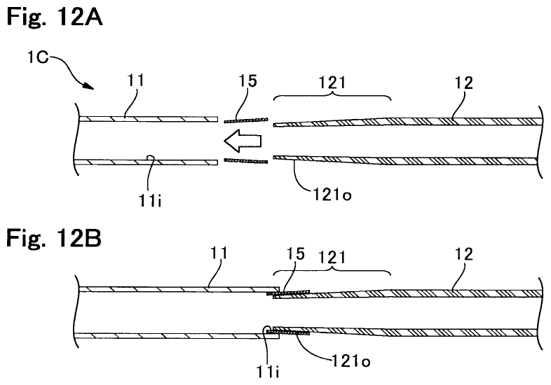

FIGS. 12A and 12B are diagrams illustrating a method for manufacturing a catheter 1C according to a fourth embodiment. The catheter 1C according to the fourth embodiment differs from that according to the first embodiment described in FIGS. 9A to 9C in the content of the insertion step. According to the fourth embodiment, the constriction formation step, the tapered portion formation step, and the joining step are the same as those of the first embodiment.

FIG. 12A illustrates a state before insertion in the insertion step. As illustrated in FIG. 12A, the operator prepares the first distal end side tube 11 and a connection member 15. The connection member 15 is a member that is provided between the first distal end side tube 11 and the first proximal end side tube 12 to connect these tubes. In the illustrated example, the connection member 15 is a tubular body having a tapered shape in which the outer diameter and the inner diameter are reduced from the proximal end toward the distal end. As the connection member 15, for example, a PTFE tube can be used. As indicated by the white arrow in FIG. 12A, the operator inserts the connection member 15 into the first distal end side tube 11 and inserts the first proximal end side tube 12 into the connection member 15.

FIG. 12B illustrates a state after insertion in the insertion step. As a result of FIG. 12A, as illustrated in FIG. 12B, the first proximal end side tube 12 and the first distal end side tube 11 are joined to each other via the connection member 15 in an immiscible state without being in contact with each other. Specifically, the outer peripheral surface 121o of the first tapered portion 121 of the first proximal end side tube 12 is in contact with an inner peripheral surface of the connection member 15. The inner peripheral surface 11i of the first distal end side tube 11A is in contact with the outer peripheral surface of the connection member 15. The subsequent procedure is the same as described in the first embodiment.

As described above, the method for manufacturing the catheter 1C can be variously changed, and the first proximal end side tube 12 and the first distal end side tube 11 may be joined to each other in an immiscible state without being in contact with each other by using the connection member 15. The above-described change is merely an example, and the method for manufacturing the catheter 1C can be changed in various ways. For example, in the joining step, the adhesive may be applied to at least one of the outer peripheral surface of the connection member 15 and the outer peripheral surface of the first tapered portion 121. The catheter 1C and the method for manufacturing the catheter 1C according to the fourth embodiment described above can also achieve the same effects as those of the first embodiment described above.

### Fifth Embodiment

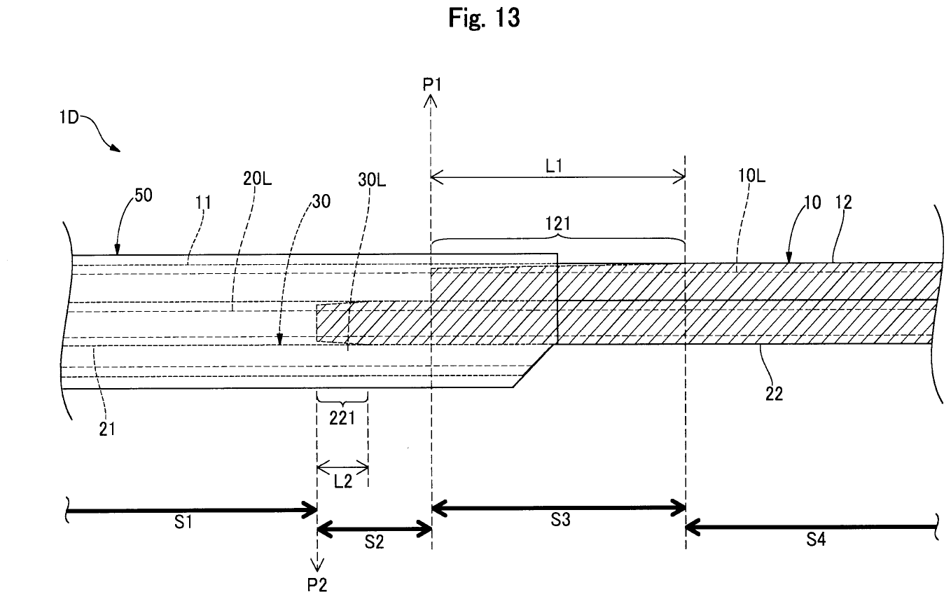

FIG. 13 is an enlarged view of the vicinity of a tube joint part in a catheter 1D according to a fifth embodiment. The catheter 1D according to the fifth embodiment does not include the second outer tube 80 and the heat-shrinkable tube 90 in the configuration described in the first embodiment. According to the fifth embodiment, on the proximal end side with respect to the first outer tube 50, the first proximal end side tube 12 and the second proximal end side tube 22 are joined to each other by thermal welding or any joining material (e.g., metal solder such as silver solder, gold solder, zinc, Sn-Ag alloy, or Au-Sn alloy, or an adhesive such as an epoxy-based adhesive).

As described above, the configuration of the catheter 1D can be variously changed, and at least any one of the second outer tube 80 and the heat-shrinkable tube 90 may be omitted. The catheter 1D according to the fifth embodiment described above can also achieve the same effects as those of the first embodiment described above.

### Modification of Present Embodiment

The disclosed embodiments are not limited to the above-described embodiments, and can be implemented in various modes without departing from the gist of the disclosed embodiments, and for example, the following modifications are also possible.

### Modification 1

In the first to fifth embodiments, examples of the configurations of the catheters 1 and 1A to 1D have been described. However, the configurations of the catheters 1 and 1A to 1D can be variously changed.

For example, the distal end positions P1 and P2 in the longitudinal direction of the catheter 1 may be reversed. Specifically, the distal end position P2 of the second proximal end side tube 22 may be closer to the proximal end side than the distal end position P1 of the first proximal end side tube 12. For example, the RX tube 30 may be omitted, and the catheter 1 may be configured to have two lumens. For example, the outer diameter of the first proximal end side tube 12 and the outer diameter of the second proximal end side tube 22 may be equal to each other, or the size relationship of the outer diameter may be reversed from that in the first embodiment. For example, the melting point of the first proximal end side tube 12 may be equal to the melting point of the first distal end side tube 11, or the magnitude relationship of the melting point may be reversed from that in the first embodiment. For example, the first proximal end side tube 12 and the first distal end side tube 11 may be joined to each other in a compatible state. With respect to these points (outer diameter, melting point, compatibility), the same applies to the second distal end side tube 21 and the second proximal end side tube 22.

For example, the outer peripheral surfaces of the first outer tube 50, the heat-shrinkable tube 90, and the second outer tube 80 or the outer peripheral surface of the catheter 1 including these may be coated with a hydrophilic resin or a hydrophobic resin.

For example, the sensor 70 is incorporated in the sensor lumen 10L of the sensor tube 10 and is not removable from the catheter 1. However, the sensor 70 may be configured to be removable from the catheter 1. That is, the catheter 1 may omit the sensor 70 as a constituent element.

For example, at least one of the distal tip 40, the first marker 41, and the second marker 42 may be omitted. For example, the shapes of the distal tip 40, the first marker 41, and the second marker 42 can be arbitrarily changed. The distal tip 40 may have a constant outer diameter from the distal end toward the proximal end, and the shape of the transverse section may be a non-circular symmetric shape. The first marker 41 and the second marker 42 may have a shape different from the annular shape (for example, a shape obtained by cutting an annular ring at an arbitrary angle, a linear shape, or a coil shape obtained by spirally winding a wire).

For example, the arrangement of the distal tip 40, the first marker 41, and the second marker 42 can be arbitrarily changed. The first marker 41 does not need to overlap with the distal tip 40, and may be provided at a position adjacent to the proximal end of the distal tip 40 or at a position away from the proximal end of the distal tip 40. The second marker 42 may be provided at a position (for example, a position away from the distal end opening 201) different from the position adjacent to the distal end of the distal end opening 201 of the OTW tube 20. The first marker 41 and the second marker 42 may be provided on a tube (the sensor tube 10 or the OTW tube 20) different from the RX tube 30. The first marker 41 and the second marker 42 may be provided on the same tube as described above, or may be provided on different tubes.

For example, at least one of the first outer tube 50 and the second outer tube 80 may be omitted. For example, in the example of the embodiment described above, each of the first outer tube 50 and the second outer tube 80 is formed of one layer, but at least one of the first outer tube 50 and the second outer tube 80 may be formed of two or more layers. For example, in the example of the embodiment described above, the transverse sectional shape of the portion of the catheter 1 covered with the first outer tube 50 is a triangular shape with round corners, but may be any shape such as a circular shape or an elliptical shape. For example, in the example of the embodiment described above, the transverse sectional shape of the portion of the catheter 1 covered with the second outer tube 80 is an elliptical shape, but may be any shape such as a circular shape or a triangular shape with round corners. For example, at least one of the first outer tube 50 and the second outer tube 80 may have an outer shape along the contour of the outer peripheral surface of the tubes 10 and 20 (or the tubes 10, 20, and 30).

For example, the shapes of the branch connector 60, the first reinforcing member 61 to the third reinforcing member 63, the cylindrical member 64, the connector 65, and the connector 25 described above are merely examples, and may be arbitrarily changed. For example, at least a part of the branch connector 60, the first reinforcing member 61, the second reinforcing member 62, and the cylindrical member 64 may be configured as a single member or may be omitted. For example, the third reinforcing member 63 and the connector 25 may be configured as one member. For example, the cylindrical member 64 may include a mechanism (e.g., a scale or a stopper provided for each predetermined length in the longitudinal direction, or a scale or a stopper provided for each predetermined angle in the circumferential direction) that assists adjustment of at least one of the front-back position of the sensor 70 and the orientation of the sensor 70 in the circumferential direction.

For example, the methods for manufacturing the catheters 1 and 1A to 1D described in FIGS. 8A to 8C, 9A to 9C, 10A to 10C, 12A, and 12B are merely examples, and various changes can be made. For example, the heating temperature in the constriction formation step can be arbitrarily changed as long as it is a temperature at which the tube 12a is melted and deformed, and does not need to be a temperature higher than the melting point of the first distal end side tube 11 by 100° C. or more. For example, pretreatment for processing or a separate step for arranging other members which are not described may be further provided between the above-described steps.

### Modification 2

The configurations of the catheters 1 and 1A to 1D according to the first to fifth embodiments and the configurations of the catheters 1 and 1A to 1D according to the modification 1 may be combined as appropriate. For example, the omission of the second outer tube 80 or the heat-shrinkable tube 90 described in the fifth embodiment may be combined with the catheters 1A and 1C configured by the method described in any one of the second and fourth embodiments. For example, the omission of the second outer tube 80 or the heat-shrinkable tube 90 described in the fifth embodiment may be combined with the catheter 1B described in the third embodiment.

### Aspects

(1) According to an aspect of the disclosed embodiments, a medical device is provided. The medical device includes a first proximal end side tube that has a first bending rigidity and forms a part of a first lumen, a first distal end side tube that has a second bending rigidity lower than the first bending rigidity, is provided closer to a distal end side than the first proximal end side tube, has a proximal end portion thereof joined to a distal end portion of the first proximal end side tube, and forms a part of the first lumen, a second proximal end side tube that has a third bending rigidity and forms a part of the second lumen, and a second distal end side tube that has a fourth bending rigidity lower than the third bending rigidity, is provided closer to the distal end side than the second proximal end side tube, has a proximal end portion thereof joined to a distal end portion of the second proximal end side tube, and forms a part of the second lumen, and a distal end position of the second proximal end side tube is located closer to the distal end side than a distal end position of the first proximal end side tube in a longitudinal direction of the medical device.

With this configuration, the distal end position of the second proximal end side tube is located closer to the distal end side than the distal end position of the first proximal end side tube. Specifically, a first joint part where the first distal end side tube and the first proximal end side tube are joined to each other and a second joint part where the second distal end side tube and the second proximal end side tube are joined to each other are shifted in the longitudinal direction of the medical device (located at different positions).

For this reason, as compared with the case where the first joint part and the second joint part are at the same position, it is possible to realize gradual changes in rigidity of the medical device, and it is possible to suppress the occurrence of kink in the medical device. As a result, when a pushing force is applied to the medical device during use of the medical device, buckling of the medical device can be suppressed.

(2) In the medical device according to the above-described aspect, the distal end portion of the first proximal end side tube may be provided with a tapered portion whose outer diameter becomes smaller from a proximal end toward a distal end, and an outer peripheral surface of the tapered portion may be in contact with an inner peripheral surface of the first distal end side tube. With this configuration, the first proximal end side tube can be easily connected to the first distal end side tube with the tapered portion.

(3) The medical device according to the above-described aspect may further include a third tube that forms a third lumen and has a proximal end thereof located between the distal end and the proximal end of the tapered portion. With this configuration, the medical device can be configured to have the third lumen.

(4) In the medical device according to the above-described aspect, the first proximal end side tube and the second proximal end side tube may be bundled by a heat-shrinkable tube, and the heat-shrinkable tube may be provided between the distal end and the proximal end of the tapered portion. With this configuration, by using the heat-shrinkable tube, the first proximal end side tube and the second proximal end side tube can be bundled without being bonded. In addition, since the heat-shrinkable tube is provided between the distal end and the proximal end of the tapered portion, it is possible to suppress an increase in the outer diameter of the medical device due to the presence of the heat-shrinkable tube.

(5) In the medical device according to the above-described aspect, the tapered portion may be a first tapered portion, the distal end portion of the second proximal end side tube may be provided with a second tapered portion whose outer diameter becomes smaller from a proximal end toward a distal end, an outer peripheral surface of the second tapered portion may be in contact with an inner peripheral surface of the second distal end side tube, and a length of the first tapered portion in the longitudinal direction of the medical device may be longer than a length of the second tapered portion. With this configuration, the second proximal end side tube can be easily connected to the second distal end side tube with the second tapered portion. Further, since the length of the first tapered portion and the length of the second tapered portion are different from each other, gradual changes in rigidity can be further improved.

(6) In the medical device according to the above-described aspect, an outer diameter of the first proximal end side tube may be larger than an outer diameter of the second proximal end side tube, and, in the longitudinal direction of the medical device, a proximal end position of the second tapered portion may be located closer to the distal end side than a distal end position of the first proximal end side tube. With this configuration, it is possible to reduce the variation in the outer diameter between the first proximal end side tube and the second proximal end side tube in the section in the longitudinal direction in which the three tubes (specifically, the first proximal end side tube, the second proximal end side tube, and the third tube) are present. As a result, deviation of the position of the third tube can be reduced.

(7) In the medical device according to the above-described aspect, a melting point of the first proximal end side tube may be higher than a melting point of the first distal end side tube. With this configuration, it is possible to join tubes having different melting points to each other.

(8) In the medical device according to the above-described aspect, the first proximal end side tube and the first distal end side tube may be in contact with each other in an immiscible state. With this configuration, the tubes can be joined to each other without being compatibilized with each other.

(9) In the medical device according to the above-described aspect, an outer diameter of the first proximal end side tube may be larger than an outer diameter of the second proximal end side tube.

(10) According to an aspect of the disclosed embodiments, a method for manufacturing a medical device is provided. The method for manufacturing the medical device includes stretching a first proximal end side tube to form a constriction in the first proximal end side tube, cutting the first proximal end side tube at the constriction to form a tapered portion at a distal end portion of the first proximal end side tube, and inserting at least a part of the tapered portion of the first proximal end side tube into a first distal end side tube. With this manufacturing method, the first proximal end side tube and the first distal end side tube can be joined to each other by using the tapered portion formed at the distal end portion of the first proximal end side tube.

(11) The method for manufacturing the medical device according to the above-described aspect may include, when stretching the first proximal end side tube, heating the first proximal end side tube at a temperature higher than a melting point of the first distal end side tube by 100° C. or more. With this manufacturing method, even when the melting points of the first proximal end side tube and the first distal end side tube are different from each other by 100° C. or more, the first proximal end side tube and the first distal end side tube can be joined to each other.

(12) In the method for manufacturing the medical device according to the above-described aspect, before the inserting, an outer diameter of the first distal end side tube at a proximal end may be substantially the same as an outer diameter of the first distal end side tube at a prescribed position away from the proximal end in a long axis direction, and before the inserting, an inner diameter of the first distal end side tube at the proximal end may be substantially the same as an inner diameter at the prescribed position. With this manufacturing method, the first proximal end side tube and the first distal end side tube can be joined to each other without performing processing (for example, flaring processing) on the first distal end side tube.

Although the present mode has been described above based on the embodiments and the modifications, the embodiment of the above-described mode is intended to facilitate understanding of the present mode and does not limit the present mode. The present mode can be modified and improved without departing from the gist and the scope of the claims, and the present mode includes equivalents thereof. In addition, when the technical features are not described as essential in the present specification, the technical features can be appropriately deleted.

What is claimed is:

1. A medical device comprising:
a first proximal end side tube that has a first bending rigidity and forms a part of a first lumen;
a first distal end side tube that has a second bending rigidity lower than the first bending rigidity, is provided closer to a distal end side than the first proximal end side tube, has a proximal end portion thereof joined to a distal end portion of the first proximal end side tube, and forms a part of the first lumen;
a second proximal end side tube that has a third bending rigidity and forms a part of the second lumen; and
a second distal end side tube that has a fourth bending rigidity lower than the third bending rigidity, is provided closer to the distal end side than the second proximal end side tube, has a proximal end portion thereof joined to a distal end portion of the second proximal end side tube, and forms a part of the second lumen, wherein a distal end position of the second proximal end side tube is located closer to the distal end side than a distal end position of the first proximal end side tube in a longitudinal direction of the medical device.

2. The medical device according to claim 1, wherein
the distal end portion of the first proximal end side tube is provided with a tapered portion whose outer diameter becomes smaller from a proximal end toward a distal end, and an outer peripheral surface of the tapered portion is in contact with an inner peripheral surface of the first distal end side tube.

3. The medical device according to claim 2, further comprising:
a third tube that forms a third lumen and has a proximal end thereof located between the distal end and the proximal end of the tapered portion.

4. The medical device according to claim 2, wherein
the first proximal end side tube and the second proximal end side tube are bundled by a heat-shrinkable tube, and
the heat-shrinkable tube is provided between the distal end and the proximal end of the tapered portion.

5. The medical device according to claim 2, wherein
the tapered portion is a first tapered portion,
the distal end portion of the second proximal end side tube is provided with a second tapered portion whose outer diameter becomes smaller from a proximal end toward a distal end, and an outer peripheral surface of the second tapered portion is in contact with an inner peripheral surface of the second distal end side tube, and
a length of the first tapered portion in the longitudinal direction of the medical device is longer than a length of the second tapered portion.

6. The medical device according to claim 5, wherein
an outer diameter of the first proximal end side tube is larger than an outer diameter of the second proximal end side tube, and
in the longitudinal direction of the medical device, a proximal end position of the second tapered portion is located closer to the distal end side than a distal end position of the first proximal end side tube.

7. The medical device according to claim 1, wherein
a melting point of the first proximal end side tube is higher than a melting point of the first distal end side tube.

8. The medical device according to claim 1, wherein
the first proximal end side tube and the first distal end side tube are in contact with each other in an immiscible state.

9. The medical device according to claim 1, wherein
an outer diameter of the first proximal end side tube is larger than an outer diameter of the second proximal end side tube.

10. The medical device according to claim 1, further comprising a third tube defining a third lumen, the third tube extending from the distal end side to a proximal end opening located longitudinally between the distal end position of the first proximal end side tube and the distal end position of the second proximal end side tube.

11. The medical device according to claim 1, further comprising an outer tube covering at least a portion of the first and second proximal end side tubes and at least a portion of the first and second distal end side tubes.

12. A method for manufacturing a medical device comprising:
stretching a first proximal end side tube to form a constriction in the first proximal end side tube;
cutting the first proximal end side tube at the constriction to form a tapered portion at a distal end portion of the first proximal end side tube; and
inserting at least a part of the tapered portion of the first proximal end side tube into a first distal end side tube.

13. The method for manufacturing the medical device according to claim 12, comprising, when stretching the first proximal end side tube, heating the first proximal end side tube at a temperature higher than a melting point of the first distal end side tube by 100° C. or more.

14. The method for manufacturing the medical device according to claim 12, wherein
before the inserting, an outer diameter of the first distal end side tube at a proximal end is substantially the same as an outer diameter of the first distal end side tube at a prescribed position away from the proximal end in a long axis direction, and
before the inserting, an inner diameter of the first distal end side tube at the proximal end is substantially the same as an inner diameter at the prescribed position.

15. The method according to claim 10, further comprising:
stretching a second proximal end side tube to form a constriction therein;
cutting the second proximal end side tube at the constriction to form a second tapered portion at a distal end portion thereof; and
inserting at least a part of the second tapered portion into a second distal end side tube.

16. The method according to claim 15, further comprising arranging the first proximal end side tube and the second proximal end side tube adjacent to each other such that the tapered portion and the second tapered portion are longitudinally staggered.

17. A medical device, comprising:
a body having a longitudinal axis, a proximal end, and a distal end, the body defining a first lumen and a second lumen extending therethrough, the body including a transition region located between the proximal end and the distal end, the transition region having a stepped increase in bending rigidity from the distal end to the proximal end;
wherein, within the transition region:
the first lumen is formed by a first proximal tube joined to a first distal tube at a first joint, the first proximal tube having a higher bending rigidity than the first distal tube; and
the second lumen is formed by a second proximal tube joined to a second distal tube at a second joint, the second proximal tube having a higher bending rigidity than the second distal tube,
wherein the first joint and the second joint are staggered relative to one another along the longitudinal axis.

18. The medical device according to claim 17, wherein the first proximal tube comprises a first tapered portion at the first joint and the second proximal tube comprises a second tapered portion at the second joint.
